(19) United States
(12) Patent Application Publication
(10) Pub. No.: US 2011/0020475 A1
(43) Pub. Date: Jan. 27, 2011

(54) CARDIOPLEGIC SOLUTION

(75) Inventors: Erich Gygax, Bern (CH); Thierry Carrel, Bern (CH); Hendrik Tevaearai, Bern (CH)

Correspondence Address: NIXON & VANDERHYE, PC, 901 NORTH GLEBE ROAD, 11TH FLOOR, ARLINGTON, VA, 22203, US

(73) Assignee: UNIVERSITAT BERN, Bern (CH)

(21) Appl. No.: 12/281,671

(22) PCT Filed: Mar. 14, 2007
(86) PCT No.: PCT/IB2007/050878

§ 371 Date: Nov. 14, 2008

(30) Foreign Application Priority Data
Mar. 15, 2006 (IB) 2006/050815

(51) Int. Cl. A61K 33/14; A61P 41/00

(52) U.S. Cl. 424/679

(57) ABSTRACT

The present invention relates to a new cardioplegic solution to arrest and protect the heart muscle during surgery. The cardioplegic solution according to the invention offers several significant advantages as compared to standard available solutions, and contains magnesium sulfate heptahydrate, potassium chloride, procaine hydrochloride and xylitol.

### FIELD OF THE INVENTION

The invention relates to cardiac surgery. It more precisely concerns a cardioplegic solution which can be used during cardiac surgery.

### STATE OF THE ART

When cardiac surgery started in the 1960's, pioneer surgeons tried several strategies to support the patient's circulation on one side and to protect the heart on the other side. The protective role of cold was immediately recognized for the heart but also for the whole body. Subsequent development of cardiac surgery largely took advantage of the progressive improvement in extracorporeal circuit components allowing more and more complex and prolonged procedures. A better protection than only cooling the heart muscle was thus necessary.

Understanding the cardiac cell metabolism helped developing strategies to protect the cardiac tissue. Cardioplegia, from a surgical point of view, is an induced cardiac arrest achieved by a solution perfused through the cardiac vessels, either in an antegrade way through the coronary arteries, or retrogradely through the coronary veins. From a physiologic point of view, it represents a possibility to reversibly arrest the heart. Finally, from a metabolic point of view, it protects the cardiac cells again the possible damages induced by the temporary absence of oxygenation. Several types of solutions have been tried and are routinely used in cardiac surgery in the world. These are:
Crystalloïde cardioplegia
Blood cardioplegia
Cold cardioplegia
Warm cardioplegia

The following prior art disclosures relate to cardioplegic solutions:

WO 03/057206; WO 02/11741; RU 2145843; WO 99/32172; WO 96/18293; U.S. Pat. No. 5,407,793; U.S. Pat. No. 5,290,766; U.S. Pat. No. 5,139,789; U.S. Pat. No. 4,988,515; EP 0054635.

There is however a need to improve existing cardioplegic solutions. In particular, the cardiac arrest must be as short as possible. It would also be convenient to minimize the quantity of solution used in order to obtain a cardiac arrest.

### DESCRIPTION OF THE INVENTION

The inventors have surprisingly discovered that an improved cardioplegic solution can be obtained if it contains the following elements:
Magnesium sulfate heptahydrate
Potassium chloride
Procaine hydrochloride
Xylitol

Advantageously, the following quantities are used per 1000 ml:

| | | |
|---|---|---|
| Magnesium sulfate heptahydrate | 3-5 | g |
| Potassium chloride | 0-100 | mM |
| Procaine hydrochloride | 1000-5000 | mg |
| Xylitol | 0-80 | g |

In this case the solution is preferably buffered to pH 6.5.

In a preferred embodiment the following precise quantities are used per 1000 ml:

| | |
|---|---|
| Magnesium sulfate | 4 g equiv. to 162 mM (MW = 246.48) |
| heptahydrate | |
| Potassium chloride | 50 mM equiv. to 373 mg (MW = 74.55) |
| Procaine hydrochloride | 3000 mg equiv. to 11 mM (MW = 272.78) |
| Xylitol | 45 g equiv. to 296 mM (MW = 152.15) |

In this case the solution is advantageously buffered with 0.1 M sodium hydroxide to pH 6.5.

As compared to all other state of the art cardioplegic solutions, the solution according to the present invention provides in particular the following advantages:
1. Immediate cardiac arrest (10-20 seconds). All state of the art solutions need a few minutes (traditionally 5 minutes) of perfusion to allow a secured cardiac arrest and protection. The solution according to the invention allows to reduce significantly not only the operation time, but more importantly the so called “ischemic time”, i.e. the time the cardiac muscle is not perfused with fresh blood. Indeed, there is a clear correlation between the ischemic time and the post-operative damage. Therefore, the solution according to the present invention contribute to improve the overall results and in particular the early outcome of this type of surgery.
2. The total volume of the solution according to the invention may be of only 100 ml. All currently available solutions consist of at least 1 liter of cristaloïd solution. This means that a significant hemodilution happens with the consequence that the peri- and post-operative hematocrite value usually less than 0.25 and requires for foreign blood transfusion. A reduced hematocrite. The solution according to the invention reduces this hemodilution and consequently reduces the post-operative edema.
3. The solution according to the invention allows an extended effect of at least 60 minutes with one single injection. A repeated injection or a continuous perfusion is required with all other available cardioplegic solutions. Similarly to what was just described earlier, this advantage permits the reduction of the operative and ischemic times with the subsequent already mentioned benefit.
4. Simplified technique with a direct injection performed by the surgeon himself. Indeed with currently available cardioplegic solutions, a more or less sophisticated perfusion system has to be organized and typically manipulated by a third person. Reducing the maneuvers performed by other persons clearly contribute to decrease the possible so-called cardiotechnic incidents or accidents such as air embolus.
5. Finally and importantly, the solution according to the invention provides a higher myocardial protection as compared to other currently available solutions. Indeed, the inventors have found that the level of post-operative cardiac enzymes, in other words markers of cardiac cellular lesions, is reduced as compared to other cardioplegic solutions. Finally, the hearts return spontaneously much more frequently into a sinus rhythm at the end of the procedure.

### Production (Examples)

Preferably the solution is prepared at room temperature , typically between 18 and 24° C. The preparations are considered to be safely usable when kept at 4° C. for a maximum period of 30 days.

In a preferred embodiment the substances included in the solution are mixed together in non-labeled 50 ml ready-to-use syringes.

It has been found that a stable solution over a period of at least 6 to 12 months can be achieved if sodium citrate is added.

For instance, for the preferred embodiment cited above, 160 mmol/l of sodium and 53.2 mmol/l of citrate may be used. Sterilization may be carried out during 20 minutes at 120° C.

### Surgical Procedure (Example)

Typically after median sternotomy and canulation of the heart and its great vessels, the CPB is initiated and conducted at a 100% flow rate. After verifying that the cardiac cavities are correctly unloaded, the ascending aorta is cross-clamped and the cardioplegic solution is injected into the aortic root. The heart immediately stops and the cardiac procedure can start immediately thereafter. Exceptionally, the content of a third 50 ml syringe needs to be injected, typically in patients with a higher BMI. Topic cooling is not excluded. At the end of the procedure, no hot shot is required before the aorta is declamped.

### Clinical Experience

The cardioplegic solution according to the invention was tested in several patients. The inventors did not record any single adverse effect that could be related to this solution. As compared to cardioplegic solutions of the state of the art, the solution according to the invention appears very efficient in terms of time to total cardiac arrest (immediate) and protection. The post-operative recovery is accelerated. The cardioplegic solution according to the invention may advantageously be used for coronary artery bypass procedures performed with a MECC (mini-ECC), a circuit designed to minimize the adverse effects of a standard cardiopulmonary bypass by reducing for example the priming volume (volume required to fill the system before it is connected to the patient) as well as the inflammatory reactions induced by the contact of blood with foreign materials (oxygenator, heat exchanger, tubings, filters, etc . . . ). In other procedures such as in valve operations for example, where the heart has to be widely open, the cardioplegia is initiated by the initial administration of 100 ml of the cardioplegic solution according to the invention followed by the traditional 5 minutes perfusion with conventional blood cardioplegic mixture. Usually, similarly to MECC-assisted operations, a single 100 ml cardioplegic injection is sufficient.

1. Cardioplegic solution comprising:
Magnesium sulfate heptahydrate
Potassium chloride
Procaine hydrochloride
Xylitol

2. Cardioplegic solution according to claim 1 wherein the following quantities are used per 1000 ml:

| | | |
|---|---|---|
| Magnesium sulfate heptahydrate | 3-5 | g |
| Potassium chloride | 0-100 | mM |
| Procaine hydrochloride | 1000-5000 | mg |
| Xylitol | 0-80 | g |

3. Cardioplegic solution according to claim 2 wherein the following precise quantities are used per 1000 ml:

| | |
|---|---|
| Magnesium sulfate heptahydrate | 4 g |
| Potassium chloride | 50 mM |
| Procaine hydrochloride | 3000 mg |
| Xylitol | 45 g |

4. Process for preparing a cardioplegic solution including a step where the following elements are mixed:
Magnesium sulfate heptahydrate
Potassium chloride
Procaine hydrochloride
Xylitol
